(19) United States
(12) Patent Application Publication
(10) Pub. No.: US 2014/0206925 A1
(43) Pub. Date: Jul. 24, 2014

(54) DEVICE PROVIDED WITH A SET OF APPLICATORS FOR CONTACT RADIOTHERAPY, AND SYSTEM COMPRISING SAID DEVICE

(71) Applicants: Jean-Pierre Gerard, Nice (FR); Keith Albert Spanswick, Nottinghamshire (GB); Serge Marcie, Saint Laurent Du Var (FR); Richard Trimaud, Vence (FR); Bernard Flipo, Nice Cedex (FR); Inès Raoust, Nice Cedex (FR)

(72) Inventors: Jean-Pierre Gerard, Nice (FR); Keith Albert Spanswick, Nottinghamshire (GB); Serge Marcie, Saint Laurent Du Var (FR); Richard Trimaud, Vence (FR); Bernard Flipo, Nice Cedex (FR); Inès Raoust, Nice Cedex (FR)

(21) Appl. No.: 14/113,332

(22) PCT Filed: Apr. 20, 2012
(86) PCT No.: PCT/EP2012/057317

§ 371 Date: Feb. 18, 2014

(30) Foreign Application Priority Data
Apr. 22, 2011 (FR) 11 53529

(51) Int. Cl. A61N 5/10

(52) U.S. Cl. CPC A61N 5/1015
USPC 600/7

(57) ABSTRACT

Disclosed is a device for contact radiotherapy, comprising a set of applicators, wherein each applicator comprises a hollow spherical head, with different diameters, the external surface of which is able to be in contact with a cavity of a living tissue and the internal surface of which defines an internal space able to receive a source of x-rays, with at least one area of the head is intended to have x-rays pass through it, and the thickness of said area depends on the diameter of the head configured so that the dose of x-rays produced at the external surface is between 18 and 22 gray.

Also disclosed is a system for contact radiotherapy comprising said device.

The disclosed device and system apply particularly to per-operative radiotherapy, in that the irradiation is produced in contact with the ablation bed of the tumour, in particular in the breast, during the operation.

The present invention concerns a device provided with a set of applicators for contact radiotherapy and a system comprising said device.

Contact radiotherapy is a low-voltage photon irradiation technique. It is in particular intended for the irradiation of cutaneous, superficial and endocavitary tumours.

The present invention applies more particularly to operative radiotherapy, that is to say the irradiation is carried out in contact with the ablation bed of the tumour, in particular in the breast, during the operation. For this purpose, the device comprises an applicator that is intended to be in contact with the tissue to be treated. Although the tumour has been removed, cancerous cells may be dispersed in the surrounding close tissues. Irradiation of these sterilises any cancerous cells and limits relapses.

The document US-A1-2005/0240073 is known, which describes a device for radiotherapy the applicator of which comprises a rigid body having a head, which may be made from Teflon®, substantially spherical and solid. The head comprises at least one channel with a small cross section for guidance during the insertion of the source of treatment agent.

In contact radiotherapy, the irradiation is of low voltage with very superficial action. This low-voltage irradiation combined with a solid head limits the dose produced per unit of time, that is to say the dose rate, and increases the treatment durations accordingly. In addition, the irradiation is carried out in the immediate vicinity of healthy tissues with very high doses. It is therefore particularly necessary to provide a device capable of providing controlled secure irradiation over a given area, whatever the patient and the area to be irradiated, so as to limit the risks of errors by the operators.

To this end, one aspect of the invention concerns a device provided with a plurality of applicators with a substantially spherical shape and different diameters so as to adapt to all cavities to be irradiated. The thickness of the applicator depends on the diameter of the applicator, such that it allows the delivery of a constant even dose, of around 18 to 22 gray, on the surface of the applicator, and optimises the efficiency in terms of depth.

In this way, whatever the size of the cavity to be irradiated, the head of the applicator is in contact with healthy tissue and a known controlled dose of actuator is delivered to the tissue to be irradiated.

Highly advantageously, the applicators according to the invention are hollow, which appreciably improves the dose rate on the surface of the applicator.

Other aims and advantages will emerge during the following description, which presents an embodiment of the invention that is illustrative but not limitative.

It should be stated first of all that the invention concerns a device for contact radiotherapy, characterised by the fact that it comprises a set of applicators in which each applicator comprises a hollow spherical head, with different diameters, the external surface of which is able to be in contact with a cavity in a living tissue and the internal surface of which defines an internal space able to receive a source of x-rays, in which at least one are of the head is intended to have x-rays pass through it and the thickness of said area depends on the diameter of the head configured so that the dose of x-rays produced on the external surface is between 18 and 22 gray.

According to a preferred but non-limitative alternative or cumulative variants, the invention is such that:
the thickness of said area of the head of each applicator is between 1 and 3.5 millimetres, preferentially between 1 and 2.5 millimetres;
the diameter of the head of each applicator is between 2 and 7 centimeters, preferentially between 2 and 6 centimeters;
the diameter of the head of each applicator corresponds to an angular sector of at least 300′;
the head each applicator comprises a plastics material able to be sterilised;
each applicator comprises a rim at the top part of the application head,
each applicator comprises a handle extending from the application head, said handle receiving the rim configured so as to be able to move in translation along the handle,
each applicator comprises means for fixing the head in the cavity of a living tissue;
the fixing means comprise a rim, provided with holes, forming a cap on head of the applicator;
the head of each applicator comprises an opening suitable for allowing the introduction of the source of x-rays in the internal space.

According to another possibility, the invention also concerns a contact radiotherapy system comprising a device as described above and a source of x-rays emitting an isotropic beam.

Advantageously, the source of x-rays is polychrome and emits a power of 30 to 80 kV, preferentially from 50 kV to 70 kV.

Advantageously, the source of x-rays is disposed at the centre of the head of the applicator.

Advantageously, the internal space in the application head is filled with gas, more preferentially air.

Advantageously, it comprises an aluminium filter arranged at the source of x-rays.

Advantageously, the filter has a thickness of between 0.2 and 2.5 millimetres, preferentially 0.3 millimetres.

Advantageously, the dose rate produced at the external surface is between 0.5 and 80 gray per minute, preferably 10 to 30 gray/min

The invention also concerns a irradiation method using the system described above, characterised in that a source of x-rays is inserted in an applicator head and then that the source of x-rays is activated in order to generate an energy of 30 to 80 kV, more precisely 50 kV to 70 kV, and a dose rate of between 0.5 and 80 gray per minute, preferably 10 to 30 gray/min, at the external surface of the application head.

The invention also concerns a treatment method by contact radiotherapy, preferentially per-operative, using the system described above, characterised in that it comprises the choice of an applicator with a diameter suited to the cavity to be treated, the positioning of the applicator head in contact with the tissues of the cavity to be treated, activation of a source of x-rays in order to generate an energy of 30 kV to 80 kV for a period of between 20 seconds and 40 minutes, preferably from 1 to 4 minutes, so as to administer a dose of between 18 and 22 gray on the external surface of the application head.

Advantageously, the dose administered at a tissue depth of 1 cm is 30%, advantageously 40%, greater than the dose on the external surface of the application head.

The accompanying drawings are given by way of examples and are not limitative of the invention. They represent only one embodiment of the invention and will make it possible to understand it easily.

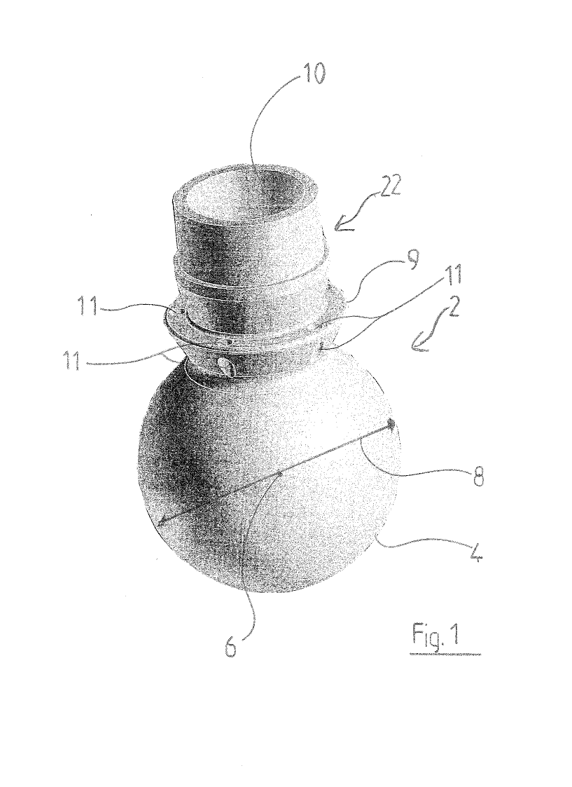

FIG. 1 is a perspective view of the applicator according to the invention.

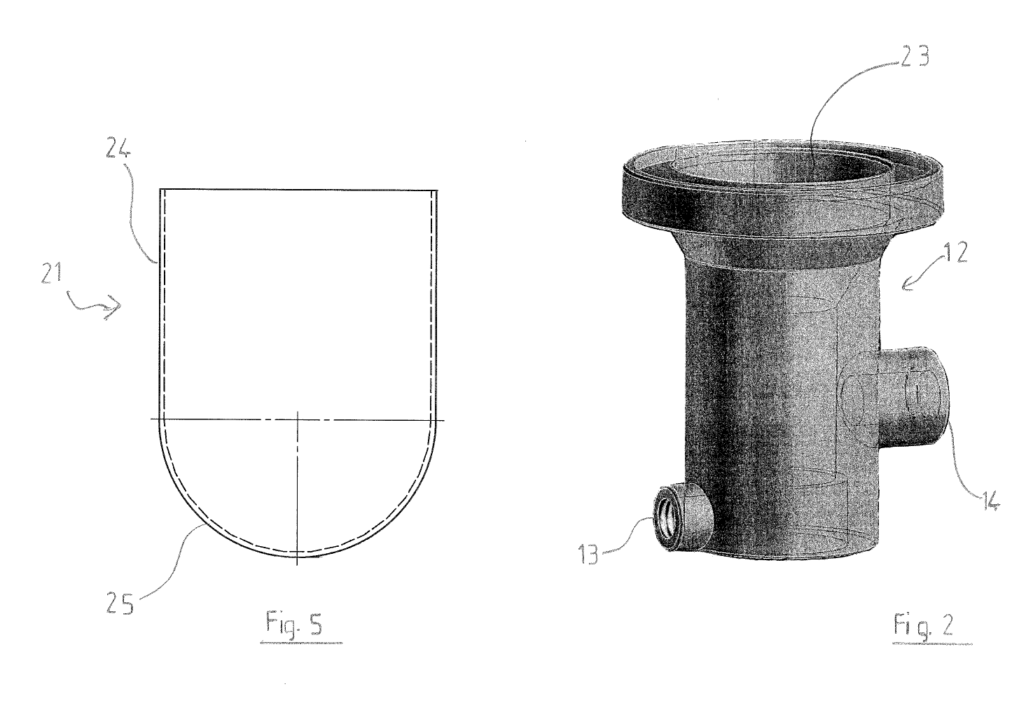

FIG. 2 illustrates an adaptor of an applicator according to the invention for connecting the applicator with a movable articulated arm.

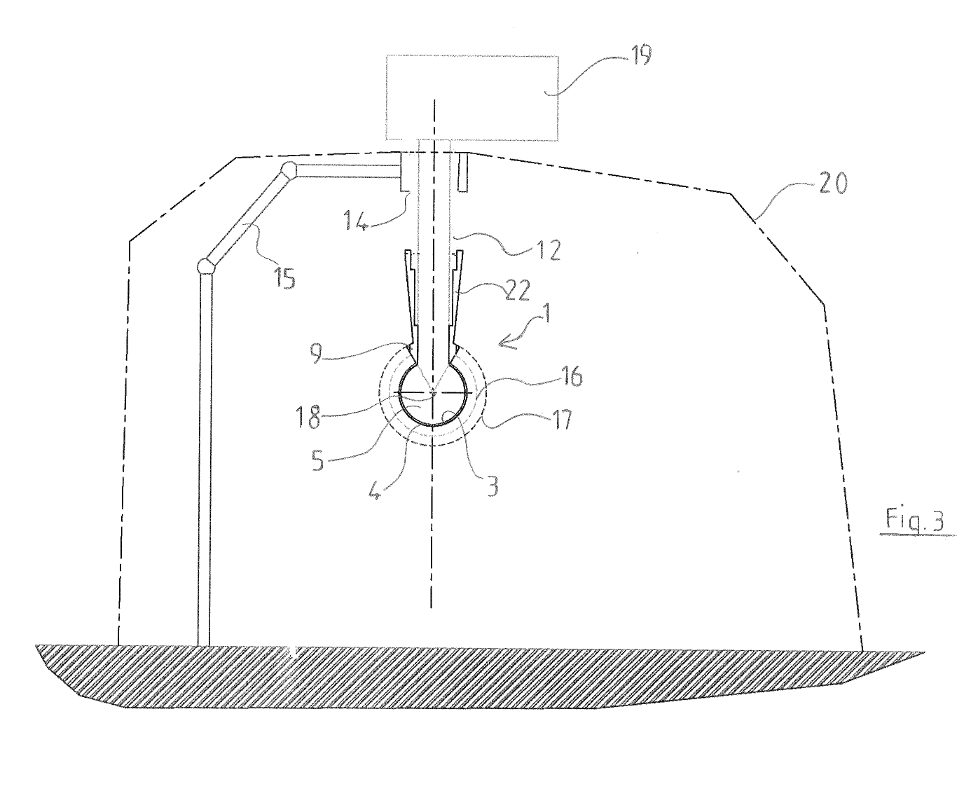

FIG. 3 shows an overall view of a per-operative contact radiotherapy system according to the invention.

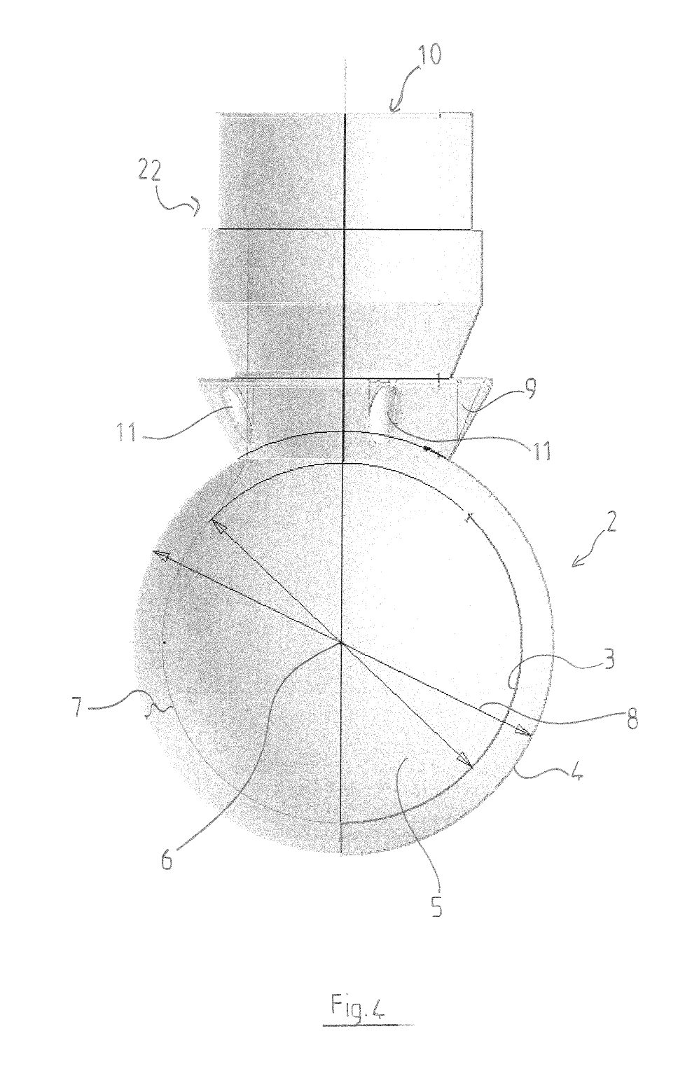

FIG. 4 is a side view of the applicator according to FIG. 1.

FIG. 5 illustrates a filter for the source of x-rays.

The invention concerns a set of applicators for contact radiotherapy. The set of applicators 1 comprises several applicators 1. The applicators 1 in the set are similar in shape. The description given below for an applicator 1 is advantageously applicable to all the applicators 1 in the set.

The applicator 1 comprises an application head 2. This application head 2 is intended to be in contact with a cavity of a living tissue. This is because per-operative contact radiotherapy makes it possible irradiate the peripheral tissue of a tumour that was removed during a surgical operation. The head 2 is chosen by the operator so that it is in contact with the ablation bed. The diameter 8 of the head 2 corresponding substantially to the diameter of the tumour removed and to the surrounding tissues removed. The application head 2 is formed by an external surface 4 intended to be in contact with the cavity to be irradiated and by an internal surface 3 defining an internal space 5. The various applicators 1 in the set of applicators according to the invention have different diameters.

The application head 2 is preferentially spherical. Spherical means that the external surface 4 of the head 2 corresponds substantially to at least one portion of a sphere. The head 2 is non-deformable and is rigid.

As shown in FIG. 1, the application head 3 is a sphere provided, at a top cap, with an opening 10. The application head 2 is a portion of a sphere, preferentially with an angular sector of at least 300°.

The application head 2 is advantageously hollow so as to receive a source of x-rays 18, preferentially passing through the opening 10. The application head 2 advantageously comprises in its internal space 5 a gas, more preferentially air. It is preferentially not filled with liquid or fluid such as water or another filling material. Preferentially, the source of x-rays 18 is disposed at the centre 6 of the head 2. The precision of positioning of the source of x-rays 18 is around 1 millimeter. The positioning of the source of x-rays 18 at the centre 6 of the head 2 advantageously helps to produce a homogeneous and constant dose of x-rays at the external surface 4. By way of example, according to the diameter 8 of the applicator 1, an adaptor 12, making it possible to connect the applicator 1 advantageously to an articulated fixing arm 15, has a different length. According to another alternative or additional possibility, it may be a support, preferentially called a handle 22, described below, which is of variable size according to the diameter 8 of the head 2 of the applicator 1. These means ensure the central placing of the source of x-rays 18 in the head 2 of each applicator, despite the fact that the head 2 is hollow.

Diameter 8 means the distance separating two points on the external surface 4 of the application head 2 passing through the centre 6 of the head 2.

By way of example, the diameter 8 of the applicators 1 varies between 2 and 6 centimeters.

At least one area of the head 2 is able to have the x-rays pass through it. This area is formed by a material the coefficient of absorption of which enables the x-ray beam to pass. The area of the head 2 receiving the x-rays and able to have them pass through it represents substantially an angular sector of at least 300°, preferentially 310°, of the head 2, that is to say substantially the entire spherical surface of the head 2.

According to the invention, the thickness 7 of the application head 2 and more precisely of the area of the application head 2 intended to have x-rays pass through it varies according to the diameter 8 of the head 2.

The thickness 7 means the distance separating the internal surface 3 and the external surface 4 of the applicator 1.

Preferentially, the thickness 7 varies between 1 and 3.5 millimetres, more precisely between 1 and 2.5 millimetres, whereas the diameter of the head 2 varies from 2 to 7 cm, more precisely between 2 and 6 cm; the internal space 5 has a diameter of between 1.8 and 6.5 cm.

One of the advantages of a hollow head 2, the internal space 5 of which is mainly filled with gas, in particular air, is to appreciably improve the dose rate at the external surface 4. The dose rate corresponds to the radiation dose received for a given unit of time. The higher the dose rate, the more quickly the dose to be administered is delivered and the irradiation period reduced. By way of preferred example, the dose rate is 10 to 30 gray with an x-ray source 18 of 50 kV.

The material intended to form the head 2 is advantageously a plastic having biocompatibility properties. The plastic chosen is preferentially high density. By way of example, a plastic of the polyamide type such as polyhexamethylene adipamide or Nylon® or a plastic able to be sterilised preferentially with gas. Preferably, the applicator 1, and more preferentially the head 2, is of the single-use type. According to one possibility, the head 2 of the applicator 1 is made from a transparent material so as to display its positioning in a cavity.

The material constituting the application head 2 and more specifically the area of the head 2 through which the x-rays pass advantageously has a coefficient of absorption of less than 5% of the dose rate.

The thickness 7 is preferentially different for the various applicators 1.

According to the invention, the greater the diameter 8 of the applicator 1, the smaller the thickness 7 and vice versa.

The variation in thickness 7 according to the diameter 8 is intended to control the dose of x-rays produced on the external surface 4 of the applicator 1 and also to optimise the efficiency in terms of depth. Conventionally, the smaller the diameter 8 of the head 2, the less good is the efficiency in terms of depth; by virtue of the applicator 1 according to the invention, the smaller the diameter 8, the greater the thickness 7, so as to maintain a satisfactory efficiency in terms of depth.

The device according to invention is particularly suited to contact radiotherapy. The objective is to obtain a satisfactory dose depth efficiency over substantially 1 cm of depth of tissue to be treated. The depth extends between the external surface 4 of the head, sinking into the tissue to be treated. According to the invention, it is sought to obtain a ratio between the dose on the external surface 4 of the head and the dose at 1 cm of depth greater than 0.3, more preferentially between 0.4 and 0.5. Beyond these steps, the radiation dose is minimal so as to protect the surrounding structures such as the heart, the ribs, the lung, etc.

According to the invention, whatever the cavity to be irradiated, that is to say whatever the diameter of the applicator 1, the dose of x-rays produced on the external surface 4 of the area of the head 2 is homogeneous.

The dose of x-rays produced on the external surface 4 of the application head 2 is between 18 and 22 gray, preferentially around 20 gray. The dose of x-rays means the dose that is absorbed by the tissue of the cavity in contact with the external surface 4 of the head 2.

The efficiency in terms of depth is controlled and substantially identical whatever the diameter of the applicator 1. Preferentially, for a tissue cavity, an absorbed dose of 6 to 9 gray is measured, or more precisely 7 to 9 gray, at a distance of 10 millimetres. For example, at a distance of 5 millimetres from the external surface 4 the absorbed does is 10 to 13 gray.

According to one embodiment, the thickness 7 may be constant over the entire area of the head 2 through which the x-rays pass, or may be different. If the thickness 7 varies over the area through which the x-rays pass, this variation may be effected by the addition of an extra piece in the internal space 5 or quite simply by a variation in the thickness 7. The variation in thickness 7 along the periphery of the area of the head 2 through which the x-rays pass is particularly advantageous when the source of x-rays used is not perfectly isotropic. In this case, the dose is not distributed uniformly on the external surface 4 of the application head 2. The dose-depth efficiency is also not constant. It is therefore prepared to increase or reduce the thickness 7. For a plastic head 2 having a base thickness 7 of 2.5 mm, the does would be reduced or increased by 10% per millimeter of thickness respectively added or removed.

According to one possibility, an additional shield may be placed in the internal space 5. This additional shield may be used for example to block the beam of x-rays over an angular sector corresponding for example to the direction of an organ to be preserved. The additional shield may be made from different material, including metal or plastic.

Advantageously, the internal space 5 also makes it possible to receive various means such as a thermoluminescent diode or a field effect transistor so as to measure the radiation delivered independently of the x-ray generator. With a simple measure without additional sterilisation the dose delivered is known according to the thickness 7 of the head 2 and the coefficient of absorption of the material used.

According to the invention, the source of x-rays 18 is isotropic; preferentially the source of x-rays 18 emits in the three dimensions over an angular sector of at least 300°, preferentially 310°. Preferentially, the source of x-rays 18 emits a beam with a power of 50 to 60 kilovolts. The beam is preferentially polychrome.

According to one embodiment of the invention, the head 2 of the applicator 1 is connected to an adaptor 12, preferentially by a handle 22. This handle 22, preferentially in the form of a hollow cylinder, enables the source of x-rays 18 to pass. The handle 22 is connected to the head 2 at the opening 10, for example fixedly. Preferentially, at the junction between the handle 22 and the head 2, the applicator 1 comprises a rim 9. The rim 9 is advantageously circular, preferentially surrounding the opening 10. The rim 9 forms an edge. The rim 9 forms a stop at which the periphery of the tissues of the cavity to be irradiated comes into contact. This arrangement has the advantage of fixing the applicator in the cavity as well as protecting from x-rays the tissues at risk that are positioned over this rim 9. Advantageously, the rim 9 represents the angular limit of the beam of x-rays. In this way, the ablation cavity is irradiated by a homogeneous dose even close to the rim 9. According to one embodiment, the rim 9 is able to move relative to the centre of the application head 2. The rim 9 is advantageously able to move at least in translation along the handle 2. It may also move in translation about the handle 22. The mobility and translation of the rim 9 with respect to the handle 22 gives a modularity to the applicator, making it possible to press the application head 2 more deeply into the tissue to be treated; the rim 9 remaining outside the ablation cavity. This is particularly advantageous when fixing means as described below are formed at said rim 9.

Advantageously, the applicator 1 comprises fixing means intended to hold the applicator 1 in the cavity to be irradiated. The fixing means cooperate with the periphery of the cavity to be irradiated. By way of example, the fixing means comprise holes 11 through which suture threads may be passed. Preferentially, the holes 11 are formed in the rim 9.

The applicator 1 is fixed to an adaptor 12 by attachment means, in particular clamping means 13. The adaptor 12 comprises means 14 for connection to an articulated arm 15.

The adaptor 12 is hollow to enable the source of x-rays 18 to pass in a direction 23.

Advantageously, the articulated arm 15, the adaptor 12 and the applicator 1 are covered by a sterile field 20. Preferably, the sterile field 20 is perforated, for example cut with sterile scissors, to allow the tube of the generator 19 to pass in the applicator 1 without compromising the good sterility of the whole of the procedure. The x-ray generator 19 is outside the sterile field 20.

According to a preferred variant of the invention, the source of x-rays 18 comprises a filter 21 intended to filter the radiation. This filter 21 is preferentially removable. Preferably, the filter 21 is made from metal such as aluminium with a thickness of 0.2 to 2.5 millimetres, more precisely 0.2 to 0.9 millimetres, preferentially 0.3 to 0.8 millimetres. It is preferred to use a filter 21 with a thickness of less than 0.4 mm since beyond that the effect of reduction of the low-energy rays, with a long wavelength, that is to say the effect of hardening of the beam, is small and the dose rate is greatly diminished, which requires increasing the duration of exposure.

This filter 21 can be used in a system for contact radiotherapy independently of the radiotherapy device according to the invention.

This filter 21 improves the efficiency in terms of depth of the beam of x-rays 18 by absorbing the low-energy radiations. The beam of x-rays is hardened.

By way of example, the filter 21 makes it possible to obtain an efficiency in terms of depth of 50% at 7 millimetres depth of living tissue compared with 50% at 5 millimetres in the absence of the filter 21. In parallel, the dose is reduced by 30%, and it is therefore necessary to increase the duration of irradiation by 30%.

The filter 21 has a shape complementary to the source of x-rays 18. The filter 21 is in the form of a sleeve composed of a cylindrical portion 24 with an emerging end, the other end of which is closed off by a rounded piece 25, preferentially a hemispherical cap. Advantageously, the thickness of the filter 21 depends on the angle of incidence of the x-rays at its surface.

The filter 21 is particularly useful for applicators 1 with a small diameter, that is to say less than 4.5 centimeters.

By way of example, with the invention, it is possible to irradiate a cavity with a dose at the external surface 4 of the applicator of around 20 gray in one go during a period of around 1 to 4 minutes, preferentially 2 minutes, with a satisfactory efficiency in terms of depth at 10 millimetres from the external surface 4 of the applicator 2.

### REFERENCES

1. Applicator
2. Head
3. Internal surface
4. External surface
5. Internal space
6. Centre of head
7. Thickness
8. Diameter
9. Rim
10. Opening
11. Holes
12. Adaptor
13. Clamping means
14. Means of connection to the arm
15. Articulated arm
16. Area at a distance of 5 millimetres from the external surface
17. Area at a distance of 10 millimetres from the external surface
18. Source of x-rays
19. Generator
20. Sterile field
21. Filter
22. Handle
23. Direction of introduction of source of x-rays
24. Cylindrical portion
25. Rounded piece

1. Device for contact radiotherapy comprising a set of applicators wherein each applicator comprises a hollow spherical head, with different diameters, the external surface of which is able to be in contact with a cavity of a living tissue and the internal surface of which defines an internal space able to receive a source of x-rays, in which at least one area of the head is intended to have x-rays pass through it and the thickness of said area of each applicator is between 1 and 3.5 millimetres and depends on the diameter of the head configured so that the dose of x-rays produced at the external surface is between 18 and 22 gray.

2. Device according to the claim 1, wherein the thickness of said area of the head of each applicator is between 1 and 2.5 millimetres.

3. Device according to claim 1, wherein the diameter of the head of each applicator is between 2 and 7 centimeters.

4. Device according to claim 1, wherein the area of the head receiving the x-rays and able to have them pass through it represents substantially an angular sector of at least 300°.

5. Device according to claim 1, wherein the head of each applicator comprises a plastic material able to be sterilised.

6. Device according to claim 1, wherein each applicator comprises a rim at the top part of the head.

7. Device according to claim 6, wherein each applicator comprises a handle extending from the head, said handle receiving the rim configured so as to be able to move in translation along the handle.

8. Device according to claim 1, wherein each applicator comprises means for fixing the head in the cavity of a living tissue.

9. Device according to claim 8, wherein the means for fixing are formed by a rim, provided with holes, forming a cap on the head of the applicator.

10. Device according to claim 1, wherein the head of each applicator comprises an opening suitable for introducing the source of x-rays into the internal space.

11. System for contact radiotherapy, comprising a device according to claim 1, and a source of x-rays emitting an isotropic beam.

12. System according to the claim 11, wherein the source of x-rays is polychrome and emits a power of 30 kV to 80 kV, preferentially 50 kV to 70 kV.

13. System according to claim 11, wherein the source of x-rays is disposed at the centre of the head of the applicator.

14. System according to claim 11, wherein the internal space of the application head is filled with gas.

15. System according to claim 11, comprising an aluminium filter arranged at the source of x-rays.

16. System according to claim 15, wherein the filter has thickness of between 0.2 and 2.5 millimetres.

17. System according to claim 11, wherein the dose rate produced at the external surface is 0.5 to 80 gray per minute.
